(19) United States
(12) Patent Application Publication
(10) Pub. No.: US 2026/0283518 A1
(43) Pub. Date: Sep. 24, 2026

(54) BIOSIGNAL MEASUREMENT SYSTEM

(71) Applicant: Nippon Telegraph and Telephone Corporation, Tokyo (JP)

(72) Inventors: Kento Watanabe, Tokyo (JP); Kenichi Matsunaga, Tokyo (JP)

(21) Appl. No.: 18/880,425

(22) PCT Filed: Sep. 6, 2022
(86) PCT No.: PCT/JP2022/033384

§ 371 Date: Dec. 31, 2024

(51) Int. Cl. A61B 5/16; A61B 5/00; A61B 5/0531

(52) U.S. Cl. CPC A61B 5/165; A61B 5/0531; A61B 5/6824; A61B 5/6828; A61B 5/7225; A61B 5/725

(57) ABSTRACT

An embodiment is a biosignal measurement system including an electrode configured to be in contact with skin of a first part of four limbs of a measurement subject, a first controller configured to obtain an electrocardiogram signal of the measurement subject based on a biopotential detected by the electrode, a perspiration sensor configured to measure electrical characteristics derived from perspiration of a second part of the four limbs of the measurement subject, and a second controller configured to calculate a mental perspiration amount of the measurement subject based on the electrical characteristics measured by the perspiration sensor.

### CROSS-REFERENCE TO RELATED APPLICATIONS

This application is a national phase entry of PCT Application No. PCT/JP2022/033384, filed on Sep. 6, 2022, which application is hereby incorporated herein by reference.

### TECHNICAL FIELD

The present invention relates to a biosignal measurement system used for monitoring person's stress and emotions.

### BACKGROUND

In recent years, monitoring of person's stress and emotions has been studied for an application field of biosignal measurement. Various biosignals such as a brain wave, an electrocardiogram, and mental perspiration have been proposed as effective biosignals used for monitoring. Among biosignals, the electrocardiogram and the mental perspiration are relatively easy to measure, and it is expected to improve the accuracy of monitoring by simultaneously measuring the electrocardiogram and the mental perspiration.

Non Patent Literature 1 discloses a sensor system that measures an electrocardiogram by using an electrocardiograma sensor device attached to the chest of a person, and measures perspiration by using a perspiration sensor device attached to the palm.

In monitoring stress and emotions, wearing sensor devices with a large number of wires on the wearer's body or wearing measurement devices connected to these sensor devices may interfere with the monitoring result and adversely affect the monitoring result. In particular, in a sticking type sensor device such as an electrocardiogramfort caused by sticking an electrode to the chest has been considered a problem.

### CITATION LIST

### Non Patent Literature

Non Patent Literature 1: Antonio Affanni, “Wireless Sensors System for Stress Detection by Means of ECG and EDA Acquisition”, Sensors (Basel), Vol.20(7):2026, Published 2020 Apr. 4, doi:10.3390/s20072026, <https://www.ncbi.nlm.nih.gov/pmc/articles/PMC7181292/>

### SUMMARY

### Technical Problem

Embodiments of the present invention has been made to solve the above-described problems, and an object of embodiments of the present invention is to provide a biosignal measurement system capable of measuring an electrocardiogram signal and a mental perspiration amount at a site where a measurement subject feels less discomfort.

### Solution to Problem

According to embodiments of the present invention, there is provided a biosignal measurement system including: an electrode configured to be in contact with skin of a part of four limbs of a measurement subject; a first control unit configured to obtain an electrocardiogram signal of the measurement subject on the basis of a biopotential detected by the electrode; a perspiration sensor configured to measure electrical characteristics derived from perspiration of a part of the four limbs of the measurement subject; and a second control unit configured to calculate a mental perspiration amount of the measurement subject on the basis of the electrical characteristics measured by the perspiration sensor.

### Advantageous Effects

According to embodiments of the present invention, by providing the electrode in contact with the skin of a part of four limbs of the measurement subject and the perspiration sensor that measures the electrical characteristics derived from the perspiration of a part of four limbs of the measurement subject, it is possible to measure the electrocardiogram signal and the mental perspiration amount at the site where the measurement subject feels less discomfort. As a result, in embodiments of the present invention, it is possible to reduce an adverse effect on an application using the measurement result.

### BRIEF DESCRIPTION OF THE DRAWINGS

FIG. 1 is a block diagram illustrating a configuration of a biosignal measurement system according to a first embodiment of the present invention.

FIG. 2 is a block diagram illustrating a configuration of an electrocardiographic sensor control unit according to the first embodiment of the present invention.

FIG. 3 is a block diagram illustrating another configuration of an electrocardiographic sensor control unit according to the first embodiment of the present invention.

FIG. 4 is a block diagram illustrating still another configuration of a biosignal measurement system according to the first embodiment of the present invention.

FIG. 5 is a block diagram illustrating a configuration of a biosignal measurement system according to a second embodiment of the present invention.

FIG. 6 is a block diagram illustrating a configuration of an electrocardiographic sensor control unit according to the second embodiment of the present invention.

FIG. 7 is a circuit diagram illustrating a configuration of an amplification unit according to the second embodiment of the present invention.

FIG. 8 is a block diagram illustrating a configuration of an electrocardiographic sensor control unit according to the second embodiment of the present invention.

FIG. 9 is a block diagram illustrating another configuration of a biosignal measurement system according to the second embodiment of the present invention.

FIG. 10 is a block diagram illustrating a configuration example of a computer that implements the biosignal measurement systems according to first and second embodiments of the present invention.

### DETAILED DESCRIPTION OF ILLUSTRATIVE EMBODIMENTS

### First Embodiment

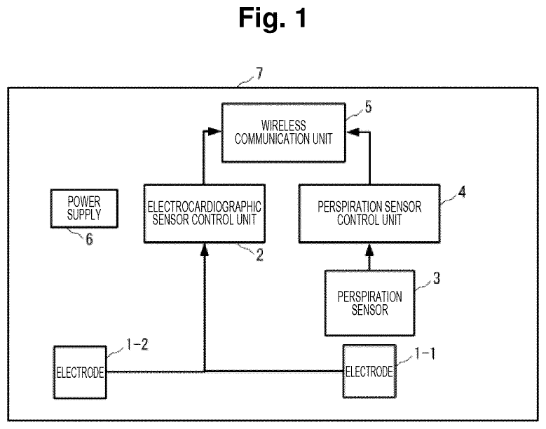

Hereinafter, embodiments of the present invention will be described with reference to the drawings. FIG. 1 is a block diagram illustrating a configuration of a biosignal measurement system according to a first embodiment of the present invention. The biosignal measurement system includes two or more electrodes 1-1 and 1-2 that are in contact with the skin of a part of four limbs of a measurement subject, an electrocardiogramsor control unit 2 (first control unit) that obtains an electrocardiogram signal of the measurement subject on the basis of biopotentials detected by the electrodes 1-1 and 1-2, a perspiration sensor 3 that measures electrical characteristics derived from perspiration of a part of four limbs of the measurement subject, a perspiration sensor control unit 4 (second control unit) that calculates a mental perspiration amount of the measurement subject on the basis of the electrical characteristics measured by the perspiration sensor 3, a wireless communication unit 5 that wirelessly transmits the electrocardiogram signal and information regarding the mental perspiration amount to the outside, and a power supply 6 that supplies power to the electrocardiogramsor control unit 2, the perspiration sensor 3, the perspiration sensor control unit 4, and the wireless communication unit 5.

The interval of an R wave, which is the peak of the electrocardiogram waveform, is affected by both the dual control of the sympathetic nerve and parasympathetic nerve of a person, and varies in a time domain and a frequency domain depending on a mental state of the person. Furthermore, mental perspiration is affected by sympathetic nerves. When a person is mentally tense, perspiration occurs on the palm and foot sole of the person. By measuring these biosignals such as the electrocardiogram and the mental perspiration in a multimodal manner, it is possible to expect more accurate mental monitoring.

In the technology disclosed in Non Patent Literature 1, the electrocardiogra sensor is attached to the chest of the measurement subject, and the perspiration sensor is attached to the palm of the hand. However, although it is convenient to attach each sensor independently, there is a possibility that strong discomfort is given to the measurement subject and mental monitoring is affected depending on the attachment site and the attachment method.

On the other hand, in the configuration illustrated in FIG. 1, the electrodes 1-1 and 1-2, the electrocardiogramsor control unit 2, the perspiration sensor 3, the perspiration sensor control unit 4, the wireless communication unit 5, and the power supply 6 are provided inside a mat-shaped sensor device 7 on which the measurement subject stand with bare feet. In a case where the electrocardiogram signal is measured as the biosignal, it is necessary to dispose a plurality of the electrodes 1-1 and 1-2 at positions between which the heart of the measurement subject is positioned, and thus the electrodes 1-1 and 1-2 are disposed so as to be in contact with the right sole and left sole of the measurement subject, respectively, at least one by one. The perspiration sensor 3 may be in contact with at least one of the right sole or the left sole.

As described above, in the present embodiment, the discomfort at the time of wearing can be reduced by making the biosignal measurement system capable of being used daily by the measurement subject, and thus, the biosignal measurement system is suitable for mental monitoring, for example, measuring the degree of tension or stress of the measurement subject, and estimating emotions.

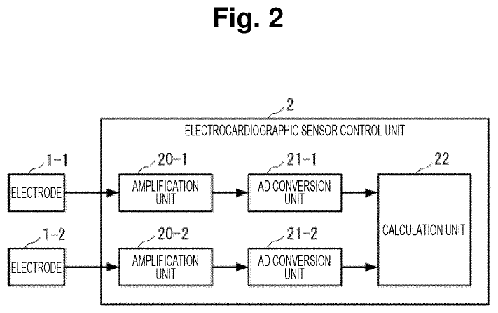

FIG. 2 is a block diagram illustrating a configuration of the electrocardiogra sensor control unit 2. The electrocardiograma sensor control unit 2 includes amplification units 20-1 and 20-2 that amplify the biopotentials detected by the electrodes 1-1 and 1-2 in contact with the skin of the measurement subject, AD conversion units 21-1 and 21-2 that convert the amplified biopotentials into digital data, and a calculation unit 22 that calculates a difference between the biopotential detected by the electrode 1-1 and the biopotential detected by the electrode 1-2 as an electrocardiogram signal.

Since the biopotentials detected by the electrodes 1-1 and 1-2 are very weak signals, signal amplification by the amplification units 20-1 and 20-2 is required. The amplification units 20-1 and 20-2 require high input impedance in order to reduce the loss of the biopotential. With an inverting amplifier circuit, the resistance for determining the input impedance also affects the gain setting, and further directly contributes as thermal noise, and thus the SN ratio of the biopotential is lowered. On the other hand, a non-inverting amplifier circuit has a feature that noise is less likely to increase even in a high input impedance configuration. Therefore, as the amplification units 20-1 and 20-2, it is effective to use the non-inverting amplifier circuits. Furthermore, low-pass filters may be provided in the amplification units 20-1 and 20-2.

The calculation unit 22 calculates a difference between the biopotential detected by the electrode 1-1 and amplified by the amplification unit 20-1 and the biopotential detected by the electrode 1-2 and amplified by the amplification unit 20-2 as an electrocardiogram signal.

As the perspiration sensor 3, there is a skin electrometer that applies a current or a voltage to an electrode in contact with the skin of the measurement subject and measures a change in impedance of the skin of the measurement subject, the change being caused by perspiration. In the case of using the skin electrometer, the perspiration sensor control unit 4 estimates the mental perspiration amount of the measurement subject on the basis of the measured change in impedance of the skin.

Furthermore, as the perspiration sensor 3, a sensor that has a structure for collecting moisture from the skin of the measurement subject and outputs an electric signal derived from the collected moisture may be used. In the case of using such a sensor, the perspiration sensor control unit 4 estimates the mental perspiration amount of the measurement subject on the basis of the electric signal output from the sensor.

In a case where the skin electrometer is used as the perspiration sensor 3, downsizing can be achieved. On the other hand, in the case of the perspiration sensor 3 that requires a structure for collecting moisture from the skin of the measurement subject, the perspiration sensor 3 is bulky, and thus may interfere with the activity of the measurement subject.

In a case where the skin electrometer is used as the perspiration sensor 3, it is not necessary to separately prepare an electrode by sharing the electrode for skin impedance measurement and the electrodes 1-1 and 1-2 for electrocardiogram signal measurement. In a case where the electrode is shared, when the signal applied to the electrode for the skin impedance measurement is a signal in the same frequency band as that of the biopotential, it is difficult to separate the signal applied to the electrode from the biopotential. Therefore, it is impossible to simultaneously perform the skin impedance measurement and the electrocardiogram signal measurement.

In order to simultaneously perform the skin impedance measurement and the electrocardiogram signal measurement, a signal to be applied to the electrode for the skin impedance measurement needs to be an AC signal having a frequency different from that of the biopotential. In this case, the potentials of the electrodes 1-1 and 1-2 include various frequency components. The electrocardiogramsor control unit 2 can extract the biopotential by frequency filtering processing for removing the signal applied by the perspiration sensor 3. The frequency filtering processing of the electrocardiogramaensor control unit 2 can be implemented by low-pass filters of the amplification units 20-1 and 20-2. Furthermore, the perspiration sensor 3 can measure the skin impedance by frequency filtering processing for removing the biopotential.

The frequency of the biopotential is approximately 50 Hz or less. The cutoff frequencies of the low-pass filters provided in the amplification units 20-1 and 20-2 are, for example, 50 Hz or 100 Hz. Therefore, the perspiration sensor 3 sets the frequency of the AC signal applied to the electrode to perform the skin impedance measurement to 100 Hz or more, and thus it is possible to separate the signal applied to the electrode from the biopotential.

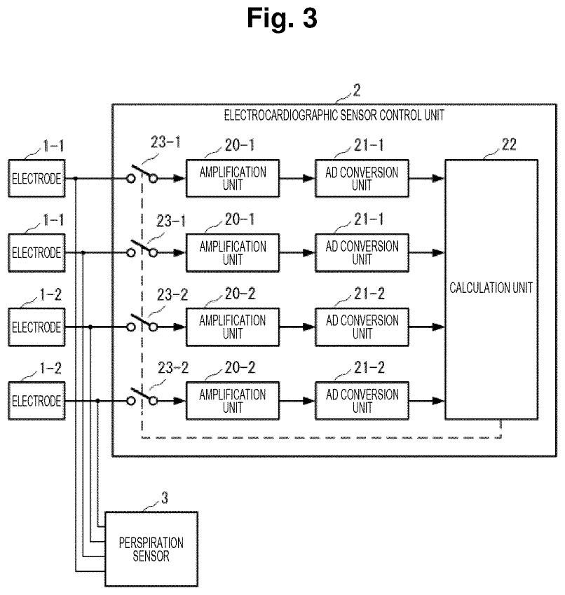

On the other hand, the skin impedance measurement and the electrocardiogram signal measurement may be performed in a time-division manner. FIG. 3 illustrates a configuration of the electrocardiograma sensor control unit 2 in a case where the skin impedance measurement and the electrocardiogram signal measurement are performed in a time-division manner. In an example of FIG. 3, switches 23-1 and 23-2 are added between the electrodes 1-1 and 1-2 and the amplification units 20-1 and 20-2. Examples of the switches 23-1 and 23-2 include a complementary metal oxide semiconductor (CMOS) switch.

The sampling rate of the electrocardiogram signal is usually 1 kHz or less, and the sampling rate of the skin impedance is about several Hz. Therefore, the switches 23-1 and 23-2 can be controlled by the calculation unit 22 including a micro controller unit (MCU). The calculation unit 22 is configured to turn off the switches 23-1 and 23-2 to connect the electrodes 1-1 and 1-2 to the perspiration sensor 3 at a timing when the perspiration sensor 3 performs the skin impedance measurement, and turn on the switches 23-1 and 23-2 to connect the electrodes 1-1 and 1-2 to the amplification units 20-1 and 20-2 at a timing when the electrocardiogram signal measurement is performed.

Note that in a case where the skin impedance measurement is performed, it is desirable to provide a plurality of electrodes at the same site of the measurement subject. For example, two or more electrodes 1-1 are provided to be in contact with the right sole of the measurement subject, and two or more electrodes 1-2 are provided to be in contact with the left sole of the measurement subject. The skin impedance of the right sole may be measured using the electrode 1-1, the skin impedance of the left sole may be measured using the electrode 1-2, or the skin impedance measurement using the electrode 1-1 and the skin impedance measurement using the electrode 1-2 may be simultaneously performed.

In the example of FIG. 3, the number of amplification units 20-1 and 20-2 and the number of AD conversion units 21-2 and 21-2 match the number of electrodes 1-1 and 1-2, but the configuration of FIG. 3 is not an essential configuration. Any configuration may be employed as long as the calculation unit 22 can calculate a difference between the biopotential detected by at least one electrode 1-1 and the biopotential detected by at least one electrode 1-2.

As the electrodes 1-1 and 1-2 and the electrode of the perspiration sensor 3, electrodes of various materials and configurations can be used. Any electrode such as an Ag/AgCl electrode used in medical applications, a cloth electrode having conductivity, or a metal electrode can be used. It is also possible to further improve the degree of freedom of the measurement subject by using a non-contact electrode configuration in which an electrode is attached to the clothing by using a cloth electrode or a metal electrode that does not need to be directly attached to the body of the measurement subject.

In the example of FIG. 1, the electrodes 1-1 and 1-2, the electrocardiogramsor control unit 2, the perspiration sensor 3, the perspiration sensor control unit 4, the wireless communication unit 5, and the power supply 6 are provided inside the mat-shaped sensor device 7. However, the biosignal measurement system may be separately attached to the right hand and left hand of the measurement subject, or may be separately attached to the right foot and the left foot.

In a case where the biosignal measurement system is separately attached to the right hand and left hand of the measurement subject, for example, it is conceivable to provide the electrodes 1-1 and 1-2, the electrocardiogramsor control unit 2, the perspiration sensor 3, the perspiration sensor control unit 4, the wireless communication unit 5, and the power supply 6 in a glove type or ring type sensor device. The measurement subject wears the biosignal measurement system by putting on gloves and puts rings on the right hand and the left hand, respectively.

In a case where the biosignal measurement system is separately attached to the right foot and left foot of the measurement subject, for example, it is conceivable to provide the electrodes 1-1 and 1-2, the electrocardiogramsor control unit 2, the perspiration sensor 3, the perspiration sensor control unit 4, the wireless communication unit 5, and the power supply 6 in a sock type sensor device or a slipper type sensor device. The measurement subject wears the biosignal measurement system by wearing socks on the right foot and the left foot or by wearing slippers on the right foot and the left foot.

Although accuracy is decreased since the detection capability for mental perspiration is reduced, the electrodes 1-1 and 1-2, the electrocardiogramsor control unit 2, the perspiration sensor 3, the perspiration sensor control unit 4, the wireless communication unit 5, and the power supply 6 may be provided in the wristband-type sensor device for convenience. The measurement subject wears the biosignal measurement system by putting wristbands on the right hand and the left hand, respectively.

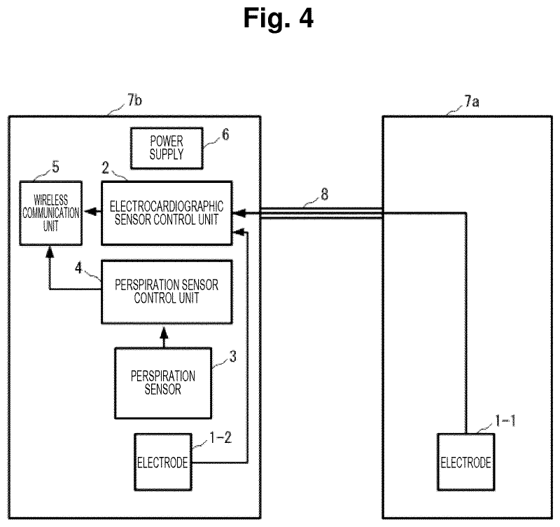

FIG. 4 illustrates a configuration of the biosignal measurement system in a case where the biosignal measurement systems are separately attached to the right side portion and the left side portion of the measurement subject. The biosignal measurement system in an example of FIG. 4 is provided separately as a sensor device 7a attached to the right side portion of the measurement subject and a sensor device 7b attached to the left side portion.

The sensor device 7a is provided with the electrode 1-1. The sensor device 7b is provided with the electrode 1-2, the electrocardiogramsor control unit 2, the perspiration sensor 3, the perspiration sensor control unit 4, the wireless communication unit 5, and the power supply 6. The electrode 1-1 and the electrocardiogramsor control unit 2 are electrically connected by a wire 8. The sensor devices 7a and 7b have the shape of a glove, a ring, a sock, a slipper, or a wristband. In a case where a cloth product such as socks or gloves is used as the base material of the sensor devices 7a and 7b, a cloth electrode using conductive fibers is particularly suitable as the electrodes 1-1 and 1-2 and the electrode of the perspiration sensor 3. Needless to say, the sensor device 7a may be attached to the left side portion of the measurement subject, and the sensor device 7b may be attached to the right side portion.

Also in the configuration of FIG. 4, the electrode of the perspiration sensor 3 for skin impedance measurement and the electrode 1-2 for electrocardiogram signal measurement can be shared.

Furthermore, as illustrated in FIGS. 1 and 4, the sensor devices 7 and 7b may be provided with the wireless communication unit 5 that wirelessly transmits the electrocardiogram signal and the information regarding the mental perspiration amount to an external device such as a smartphone or a server device.

### Second Embodiment

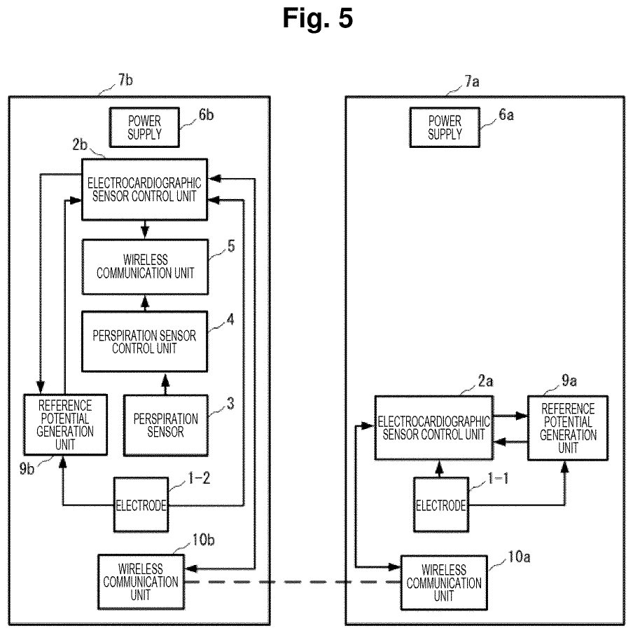

Next, a second embodiment of the present invention will be described. FIG. 5 is a block diagram illustrating a configuration of a biosignal measurement system according to a second embodiment of the present invention. In the present embodiment, as in FIG. 4, an example in which the biosignal measurement systems are separately attached to the right side portion and the left side portion of the measurement subject will be described. In the configuration of FIG. 4, since the wire 8 is used to connect the right side portion and the left side portion, there is a possibility that the activity of the measurement subject is restricted. The present embodiment solves the restriction of the activity.

The sensor device 7a attached to the right side portion of the measurement subject is provided with an electrode 1-1, an electrocardiogramsor control unit 2a, a power supply 6a, a reference potential generation unit 9a that generates a reference potential of an amplification unit of the electrocardiogramaor control unit 2a, and a wireless communication unit 10a that transmits and receives data to and from the sensor device 7b.

The power supply 6a supplies power to the electrocardiogramsor control unit 2a, the reference potential generation unit 9a, and the wireless communication unit 10a.

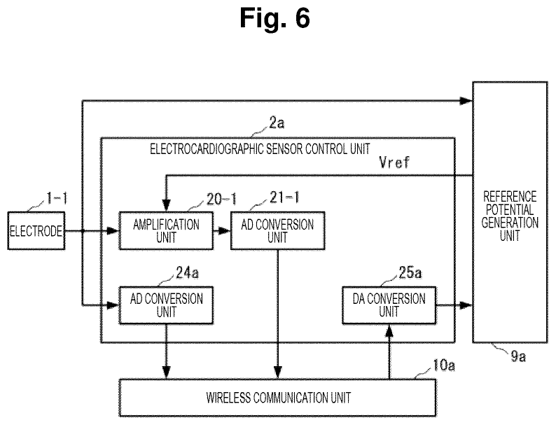

FIG. 6 is a block diagram illustrating a configuration of the electrocardiogra sensor control unit 2a. The electrocardiograma sensor control unit 2a includes an amplification unit 20-1, an AD conversion unit 21-1, an AD conversion unit 24a that converts the biopotential detected by the electrode 1-1 into digital data, and a DA conversion unit 25a that converts the digital data transmitted from the sensor device 7b and received by the wireless communication unit 10a into a biopotential.

The wireless communication unit 10a wirelessly transmits the digital data output from the AD conversion unit 24a to the sensor device 7b, and receives the digital data transmitted from the sensor device 7b. Furthermore, the wireless communication unit 10a wirelessly transmits the digital data output from the AD conversion unit 21-1 to the sensor device 7b.

In a case where non-inverting amplifier circuits are used as the amplification units 20-1 and 20-2, it is important that the reference potentials of two amplification units 20-1 and 20-2 are common. In the present embodiment, since the sensor devices 7a and 7b are not connected by a wire, the reference potentials of the amplification units 20-1 and 20-2 do not match, which may deteriorate the measurement accuracy.

In the present embodiment, in order to improve the measurement accuracy of the electrocardiogram, the reference potentials Vref in the amplification units 20-1 and 20-2 of the sensor devices 7a and 7b are made common by transmitting and receiving information regarding the biopotential between the sensor devices 7a and 7b.

As will be described later, the biopotential detected by the electrode 1-2 of the sensor device 7b is converted into digital data by an AD conversion unit 24b and transmitted from a wireless communication unit 10b of the sensor device 7b. The wireless communication unit 10a receives the digital data transmitted from the sensor device 7b. The DA conversion unit 25a converts the digital data received by the wireless communication unit 10a into a biopotential.

The reference potential generation unit 9a generates a reference potential Vref by calculating an average of addition of the biopotential detected by the electrode 1-1 and the biopotential output from the DA conversion unit 25a (biopotential detected by the electrode 1-2).

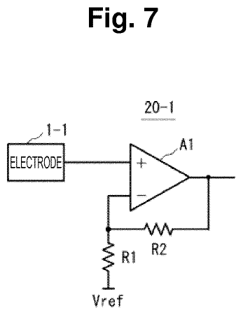

FIG. 7 is a circuit diagram illustrating a configuration example of the amplification unit 20-1. The amplification unit 20-1 includes an operational amplifier A1 and resistors R1 and R2. The reference potential Vref is supplied from the reference potential generation unit 9a to one end of the resistor R1 of the amplification unit 20-1.

On the other hand, the sensor device 7b attached to the left side portion of the measurement subject is provided with an electrode 1-2, an electrocardiogramaensor control unit 2b, a perspiration sensor 3, a perspiration sensor control unit 4, a wireless communication unit 5, a power supply 6b, a reference potential generation unit 9b that generates a reference potential of an amplification unit of the electrocardiogramsor control unit 2b, and a wireless communication unit 10b that transmits and receives data to and from the sensor device 7a. As in the example of FIG. 4, the sensor devices 7a and 7b have the shape of a glove, a ring, a sock, a slipper, or a wristband.

The power supply 6b supplies power to the electrocardiograma sensor control unit 2b, the perspiration sensor 3, the perspiration sensor control unit 4, the wireless communication unit 5, the reference potential generation unit 9b, and the wireless communication unit 10b.

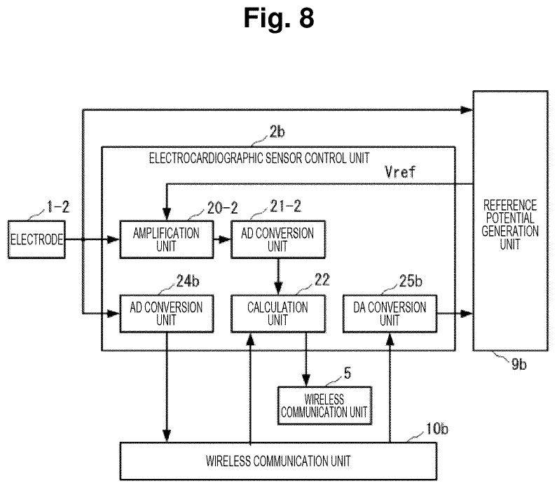

FIG. 8 is a block diagram illustrating a configuration of the electrocardiograma sensor control unit 2b. The electrocardiogramaensor control unit 2b includes an amplification unit 20-2, an AD conversion unit 21-2, a calculation unit 22, an AD conversion unit 24b that converts the biopotential detected by the electrode 1-2 into digital data, and a DA conversion unit 25b that converts the digital data transmitted from the sensor device 7a and received by the wireless communication unit 10b into a biopotential.

The wireless communication unit 10b wirelessly transmits the digital data output from the AD conversion unit 24b to the sensor device 7a. Furthermore, the wireless communication unit 10b receives data of the biopotential detected by the electrode 1-1 of the sensor device za from the sensor device 7a. The DA conversion unit 25b converts the digital data received by the wireless communication unit 10b into a biopotential.

The reference potential generation unit 9b generates a reference potential Vref by calculating an average of addition of the biopotential detected by the electrode 1-2 and the biopotential output from the DA conversion unit 25b (biopotential detected by the electrode 1-1), and supplies the reference potential Vref to the amplification unit 20-2. The configuration of the amplification unit 20-2 is similar to that of the amplification unit 20-1.

From the wireless communication unit 10a of the sensor device 7a, the biopotential before amplification and also the biopotential amplified by the amplification unit 20-1 converted into the digital data by the AD conversion unit 21-1 are wirelessly transmitted. The wireless communication unit 10b receives data of the amplified biopotential from the sensor device 7a.

The calculation unit 22 calculates a difference between the biopotential amplified by the amplification unit 20-1 of the sensor device 7a and the biopotential amplified by the amplification unit 20-2 of the sensor device 7b as an electrocardiogram signal.

Operations of the perspiration sensor 3, the perspiration sensor control unit 4, and the wireless communication unit 5 are the same as those in the first embodiment.

Needless to say, the sensor device 7a may be attached to the left side portion of the measurement subject, and the sensor device 7b may be attached to the right side portion.

As in the first embodiment, also in the present embodiment, the electrode of the perspiration sensor 3 for skin impedance measurement and the electrode 1-2 for electrocardiogram signal measurement can be shared.

As described above, in the present embodiment, the wire connecting the sensor devices 7a and 7b can be removed. Thus, the discomfort of the measurement subject, which is caused by the wire, can be reduced, and the restriction of the body of the measurement subject can be eliminated, Therefore, it is possible to measure the electrocardiogram signal and the mental perspiration amount without causing the measurement subject to be aware that the monitoring is being performed. In the present embodiment, measurement can be easily performed in daily life without hindering walking or movement of the measurement subject.

Furthermore, in the present embodiment, the common reference potentials Vref generated by the reference potential generation units 9a and 9b respectively provided in the sensor devices 7a and 7b are used. Thus, the sensor devices 7a and 7b can perform signal amplification in which the reference potentials Vref of the amplification units 20-1 and 20-2 are made common, and the measurement accuracy of the electrocardiogram signal can be improved.

Various methods can be applied as a method of wireless communication. For example, since a module for radio wave communication is widely distributed, it can be easily implemented at low cost. In a case where wireless communication using radio waves is performed, a modulation circuit and a transmission antenna are provided as transmission-side circuits of the wireless communication units 10a and 10b, and a reception antenna and a demodulation circuit are provided as reception-side circuits.

The modulation circuit on the transmission side performs digital modulation on a carrier signal by using the data to be transmitted. The modulated signal is transmitted from the transmission antenna to the sensor device on the reception side. The demodulation circuit on the reception side demodulates the signal transmitted from the sensor device on the transmission side and received by the antenna to extract data.

In a case where the wireless communication is performed using radio waves, the carrier frequencies of radio waves to be transmitted by the sensor devices 7a and 7b are set to frequencies different from each other, and thus it is possible to transmit and receive the data without interference with each other. Needless to say, the transmission antenna and the reception antenna may be shared.

Optical communication may be used as another method of transmitting and receiving the data between the sensor devices 7a and 7b. By using optical communication, it is possible to reduce the effect of the existing wireless communication widely used to stably transmit and receive data, and it is possible to expect improvement in security due to difficulty in communication interception.

In the case of using the optical communication, a modulation circuit and an electrical/optical (E/O) converter may be provided as transmission-side circuits of the wireless communication units 10a and 10b, and an optical/electrical (O/E) converter and a demodulation circuit may be provided as reception-side circuits.

The modulation circuit on the transmission side performs digital modulation on a carrier signal by using the data to be transmitted. The E/O converter converts the modulated signal into light and transmits the light to the sensor device on the reception side. The O/E converter on the reception side receives an optical signal from the sensor device on the transmission side and converts the optical signal into an electric signal. The demodulation circuit demodulates the signal output from the O/E converter and extracts data.

As in the case of using wireless communication using radio waves, by setting the wavelengths of light used in the sensor devices 7a and 7b to be different, it is possible to transmit and receive data without interference with each other.

Magnetic communication used in wireless earphones or the like is also suitable for the present embodiment. In the magnetic communication, signal transmission is performed by mutual induction with another device on the basis of a magnetic field change. The magnetic field indicates permeability to human body components such as moisture. Therefore, since communication can be performed with low interference, stable data transmission and reception can be performed even in a case where the sensor devices 7a and 7b are attached to the measurement subject.

In the case of using the magnetic communication, a modulation circuit, a voltage-current converter such as a transconductance amplifier, and a coil serving as an antenna are provided as transmission-side circuits of the wireless communication units 10a and 10b, and a coil, a current-voltage converter such as a transimpedance amplifier, and a demodulation circuit are provided as reception-side circuits.

The modulation circuit on the transmission side performs digital modulation on a carrier signal by using the data to be transmitted. The voltage-current converter converts the modulated signal into a current and supplies the current to the coil. The current supplied to the coil of the sensor device on the transmission side causes the magnetic field change. The current-voltage converter on the reception side converts the current generated in the coil on the reception side with the magnetic field change caused by the sensor device on the transmission side into a voltage. The demodulation circuit demodulates the signal output from the current-voltage converter and extracts data.

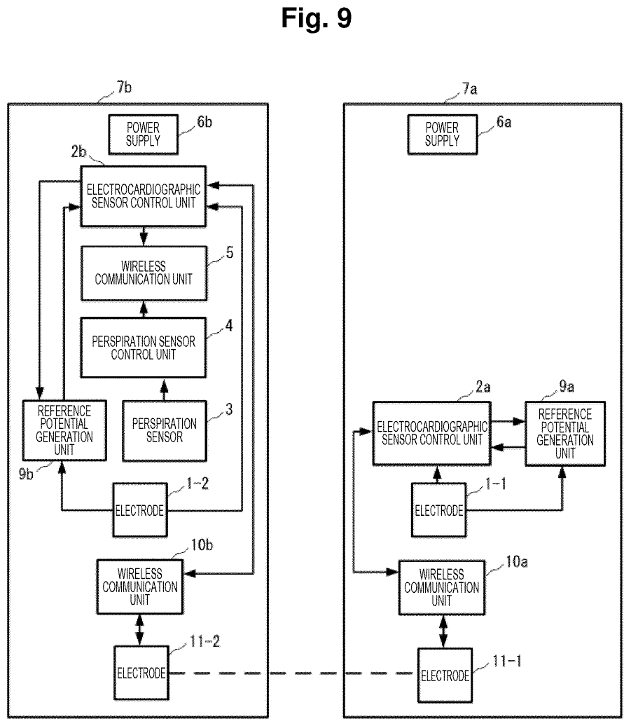

As another method of transmitting and receiving the data between the sensor devices 7a and 7b, human body communication using the body of the measurement subject as a transmission path may be used. FIG. 9 illustrates a configuration of the biosignal measurement system in this case. In the case of using the human body communication, as illustrated in FIG. 9, the sensor devices 7a and 7b include the electrodes 11-1 and 11-2, respectively, for performing the human body communication in addition to the electrodes 1-1 and 1-2.

Power for communication accounts for much of the power consumption of the sensor devices 7a and 7b. In spatial propagation using radio waves, the signal intensity attenuates in inverse proportion to the square of the propagation distance. On the other hand, in the case of the human body communication, the attenuation of the signal is inversely proportional to the propagation distance. Therefore, by using the human body communication, data transmission with less transmission power becomes possible. Transmission and reception of the data via the human body can contribute to reduction in power consumption.

The wireless communication units 10a and 10b in FIG. 9 digitally modulate the data to be transmitted into the carrier signal, and transmit the modulated signal from the electrodes 11-1 and 11-2 in contact with the skin of the measurement subject to another sensor device via the body of the measurement subject. Furthermore, the wireless communication units 10a and 10b demodulate signals transmitted from another sensor device and received by the electrodes 11-1 and 11-2, and extract data.

By using the human body communication for transmission and reception of the data, it is possible to independently set a sampling rate of a biopotential signal for generating a reference potential and a sampling rate of a biopotential signal for generating a biosignal in addition to reduction in power, and there is an advantage that the degree of freedom in design is improved.

In a case where the human body is used as a transmission path, interference can be prevented by setting the carrier frequencies used in the sensor devices 7a and 7b to different frequencies. By setting the frequency band to be used to about several MHz to about 100 MHz on the basis of the electrical properties of the human body, the human body communication with less loss can be achieved.

As illustrated in FIG. 9, the electrodes 1-1 and 1-2 and the electrodes 11-1 and 11-2 may be individually provided in each of the sensor devices 7a and 7b. Furthermore, the electrode 1-1 and the electrode 11-1 may be shared in the sensor device 7a, and the electrode 1-2 and the electrode 11-2 may be shared in the sensor device 7b.

In a case where the electrodes are shared, it is possible to separate a signal to be transmitted and received from a biopotential to be detected for electrocardiogram signal measurement by providing band pass filters having different pass bands. By reducing the number of electrodes, the number of portions in contact with the measurement subject is reduced, and thus the comfort of the measurement subject can be improved.

When the carrier signals are digitally modulated in the sensor devices 7a and 7b, multi-level modulation such as Quadrature Phase shift Keying (QPSK) is used, and different signal points used by the sensor devices 7a and 7b are set, so that it is possible to separate signals to be transmitted and received in one carrier signal. Thus, since a common device can be used as a device used for digital modulation, mass productivity and maintainability of the device can be improved.

Note that when the measured data is analyzed, a method of using the electrocardiogram signal may include heart rate variability analysis in many cases. The information required in the heart rate variability analysis is not the entire electrocardiogram waveform, and the information may be obtained by extracting a heartbeat interval as a feature. Since the heartbeat interval is an interval between peaks of the electrocardiogram waveform, the R wave having the sharpest peak in the electrocardiogram waveform may be used for detecting the heartbeat interval.

In the case of detecting the R wave, when two electrodes 1-1 and 1-2 are provided, and for example, a potential difference between two electrodes 1-1 in contact with the palm of the right hand of the measurement subject or a potential difference between two electrodes 1-2 in contact with the palm of the left hand is calculated, a signal related to the R wave can be obtained although the signal is smaller than a normal electrocardiogram signal. That is, it is possible to detect the R wave by attaching the sensor device to either the right side portion or the left side portion of the measurement subject. The reason why the R wave can be detected in either the right side portion and the left side portion is considered to be that the electric field distribution of the human body caused by the electrical activity of the heart is not uniform and slightly differs depending on the location. Therefore, the potential difference can be detected.

As described above, since the R wave can be detected even in a case where the sensor device is attached to either one of the right side portion or the left side portion of the measurement subject, the sensor device may be attached to either one of the right hand or the left hand of the measurement subject, or may be attached to either one of the right foot or the left foot. In this manner, the restriction of the body of the measurement subject can be eliminated.

As a technology similar to the electrocardiogram measurement, there is a technology of measuring a pulse interval by a pulse wave sensor. However, the pulse wave sensor has a mechanism for measuring a volume change of a blood vessel, such as a radial artery and a capillary vessel, which is close to the body surface, the volume change accompanying the heartbeat of the heart, by an optical or physical method. Therefore, there is a delay in the propagation of the volume change of the blood vessel, and the volume of the blood vessel changes depending on the movement and position of the measurement site. Therefore, the pulse interval is practically less accurate than the heartbeat interval, and is not suitable for monitoring. In embodiments of the present invention, it is possible to obtain a suitable result for monitoring stress and emotions by using the electrocardiogram measurement.

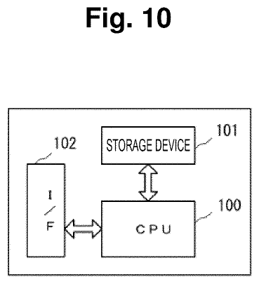

The calculation unit 22, and the perspiration sensor control unit 4 described in the first and second embodiments can be implemented by a computer that includes a central processing unit (CPU), a storage device, and an interface, and a program for controlling hardware resources. A configuration example of the computer is illustrated in FIG. 10.

The computer includes a CPU 100, a storage device 101, and an interface device (I/F) 102. The perspiration sensor 3, the wireless communication units 5 and 10b, the AD conversion units 21-1 and 21-2, and the like are connected to the I/F 102. A program for implementing the method of embodiments of the present invention is stored in the storage device 101. The CPU 100 executes the processing described in the first and second embodiments according to the program stored in the storage device 101. Furthermore, at least a part of the calculation unit 22 and the perspiration sensor control unit 4 may be configured by hardware logic such as a field-programmable gate array (FPGA).

Some or all of the above-described embodiments may be described as the following supplementary notes, but are not limited to the following.
(Supplementary note 1) A biosignal measurement system according to embodiments of the present invention includes: an electrode configured to be in contact with skin of a part of four limbs of a measurement subject; a first control unit configured to obtain an electrocardiogram signal of the measurement subject on the basis of a biopotential detected by the electrode; a perspiration sensor configured to measure electrical characteristics derived from perspiration of a part of the four limbs of the measurement subject; and a second control unit configured to calculate a mental perspiration amount of the measurement subject on the basis of the electrical characteristics measured by the perspiration sensor.
(Supplementary note 2) The biosignal measurement system according to Supplementary note 1 includes: a first sensor device configured to be attached to one of a right side portion and a left side portion of the measurement subject; and a second sensor device configured to be attached to the other one of the right side portion and the left side portion, in which the first sensor device includes a first electrode configured to be in contact with the skin of one of the right side portion and the left side portion, the second sensor device includes a second electrode configured to be in contact with the skin of the other one of the right side portion and the left side portion, the first control unit, the perspiration sensor, and the second control unit, and the first control unit obtains the electrocardiogram signal of the measurement subject on the basis of the biopotentials detected by the first and second electrodes.
(Supplementary note 3) In the biosignal measurement system according to Supplementary note 1 or 2, the perspiration sensor measures a change in impedance of the skin of the measurement subject as the electrical characteristics, and the second control unit calculates a mental perspiration amount of the measurement subject on the basis of the measured change in impedance of the skin.
(Supplementary note 4) In the biosignal measurement system according to Supplementary note 2, an electrode for the perspiration sensor to measure the electrical characteristics and the second electrode are common, the perspiration sensor applies a signal having a frequency different from that of the biopotential to the second electrode to measure the electrical characteristics, and the first control unit extracts the biopotential by frequency filtering processing.
(Supplementary note 5) In the biosignal measurement system according to Supplementary note 2, an electrode for the perspiration sensor to measure the electrical characteristics and the second electrode are common, and a measurement of the electrical characteristics by the perspiration sensor and a measurement of the electrocardiogram signal by the first control unit are performed in a time-division manner.
(Supplementary note 6) In the biosignal measurement system according to Supplementary note 5, the second sensor device further includes a switch for switching connection of the second electrode, and the first control unit controls the switch such that the second electrode and the perspiration sensor are connected at a timing when the perspiration sensor measures the electrical characteristics, and the second electrode and the first control unit are connected at a timing when the first control unit measures the electrocardiogram signal.
(Supplementary note 7) In the biosignal measurement system according to Supplementary note 2, the first sensor device further includes a first amplification unit configured to amplify the biopotential detected by the first electrode, and a first wireless communication unit configured to transmit data of the biopotential amplified by the first amplification unit to the second sensor device, the second sensor device further includes a second amplification unit configured to amplify the biopotential detected by the second electrode, and a second wireless communication unit configured to receive the data of the biopotential transmitted from the first sensor device, and the first control unit obtains an electrocardiogram signal of the measurement subject on the basis of the biopotential received by the second wireless communication unit and the biopotential amplified by the second amplification unit.
(Supplementary note 8) In the biosignal measurement system according to Supplementary note 7, the first sensor device further includes a first reference potential generation unit configured to generate a reference potential of the first amplification unit, the second sensor device further includes a second reference potential generation unit configured to generate a reference potential of the second amplification unit, the first wireless communication unit transmits, to the second sensor device, data of a biopotential before being amplified by the first amplification unit in addition to the data of the biopotential amplified by the first amplification unit, and receives the data of the biopotential transmitted from the second sensor device, the second wireless communication unit transmits, to the first sensor device, data of a biopotential before being amplified by the second amplification unit in addition to the data of the biopotential amplified by the second amplification unit, the first reference potential generation unit receives the biopotential before being amplified by the second amplification unit via the first wireless communication unit, and generates a reference potential of the first amplification unit on the basis of the biopotential before being amplified by the first amplification unit and the biopotential before being amplified by the second amplification unit, and the second reference potential generation unit receives the biopotential before being amplified by the first amplification unit via the second wireless communication unit, and generates a reference potential of the second amplification unit on the basis of the biopotential before being amplified by the second amplification unit and the biopotential before being amplified by the first amplification unit.

### Industrial Applicability

Embodiments of the present invention can be applied to a technology for measuring the electrocardiogram signal and the mental perspiration amount.

### Reference Signs List

1-1, 1-2, 11-1, 11-2 Electrode
2 Electrocardiographic sensor control unit
3 Perspiration sensor
4 Perspiration sensor control unit
5, 10a, 10b Wireless communication unit
6, 6a, 6b Power supply
7, 7a, 7b Sensor device
8 Wire
9a, 9b Reference potential generation unit
20-1, 20-2 Amplification unit
21-2, 21-2, 24a, 24b AD conversion unit
22 Calculation unit
23-1, 23-2 Switch
25a, 25b DA conversion unit

1-8. (canceled)

9. A biosignal measurement system comprising:
an electrode configured to be in contact with skin of a first part of four limbs of a measurement subject;
a first controller configured to obtain an electrocardiogram signal of the measurement subject based on a biopotential detected by the electrode;
a perspiration sensor configured to measure electrical characteristics derived from perspiration of a second part of the four limbs of the measurement subject; and
a second controller configured to calculate a mental perspiration amount of the measurement subject based on the electrical characteristics measured by the perspiration sensor.

10. The biosignal measurement system according to claim 9, comprising:
a first sensor device configured to be attached to one of a right side portion or a left side portion of the measurement subject; and
a second sensor device configured to be attached to the other one of the right side portion or the left side portion,
wherein the first sensor device includes a first electrode configured to be in contact with the skin of one of the right side portion and the left side portion,
the second sensor device includes a second electrode configured to be in contact with the skin of the other one of the right side portion and the left side portion, the first controller, the perspiration sensor, and the second controller, and
the first controller obtains the electrocardiogram signal of the measurement subject based on biopotentials detected by the first and second electrodes.

11. The biosignal measurement system according to claim 9, wherein
the perspiration sensor measures a change in impedance of the skin of the measurement subject as the electrical characteristics, and
the second controller calculates a mental perspiration amount of the measurement subject based on the measured change in impedance of the skin.

12. The biosignal measurement system according to claim 10, wherein
an electrode for the perspiration sensor to measure the electrical characteristics and the second electrode are common,
the perspiration sensor applies a signal having a frequency different from that of the biopotential to the second electrode to measure the electrical characteristics, and
the first controller extracts the biopotential by frequency filtering processing.

13. The biosignal measurement system according to claim 10, wherein
an electrode for the perspiration sensor to measure the electrical characteristics and the second electrode are common, and
a measurement of the electrical characteristics by the perspiration sensor and a measurement of the electrocardiogram signal by the first controller are performed in a time-division manner.

14. The biosignal measurement system according to claim 13, wherein
the second sensor device further includes a switch for switching connection of the second electrode, and
the first controller controls the switch such that the second electrode and the perspiration sensor are connected at a timing when the perspiration sensor measures the electrical characteristics, and the second electrode and the first controller are connected at a timing when the first controller measures the electrocardiogram signal.

15. The biosignal measurement system according to claim 10, wherein
the first sensor device further includes a first amplifier configured to amplify the biopotential detected by the first electrode, and a first wireless communicator configured to transmit data of the biopotential amplified by the first amplifier to the second sensor device,
the second sensor device further includes a second amplifier configured to amplify the biopotential detected by the second electrode, and a second wireless communicator configured to receive the data of the biopotential transmitted from the first sensor device, and
the first controller obtains an electrocardiogram signal of the measurement subject based on the biopotential received by the second wireless communicator and the biopotential amplified by the second amplifier.

16. The biosignal measurement system according to claim 15, wherein
the first sensor device further includes a first reference potential generator configured to generate a reference potential of the first amplifier,
the second sensor device further includes a second reference potential generator configured to generate a reference potential of the second amplifier,
the first wireless communicator transmits, to the second sensor device, data of a biopotential before being amplified by the first amplifier in addition to the data of the biopotential amplified by the first amplifier, and receives the data of the biopotential transmitted from the second sensor device,
the second wireless communicator transmits, to the first sensor device, data of a biopotential before being amplified by the second amplifier in addition to the data of the biopotential amplified by the second amplifier,
the first reference potential generator receives the biopotential before being amplified by the second amplifier via the first wireless communicator, and generates a reference potential of the first amplifier based on the biopotential before being amplified by the first amplifier and the biopotential before being amplified by the second amplifier, and
the second reference potential generator receives the biopotential before being amplified by the first amplifier via the second wireless communicator, and generates a reference potential of the second amplifier based on the biopotential before being amplified by the second amplifier and the biopotential before being amplified by the first amplifier.

17. The biosignal measurement system according to claim 9, wherein the first part and the second part are a same part of the four limbs.

18. A method for measuring biosignals, comprising:
contacting an electrode with skin of a first part of four limbs of a measurement subject;
obtaining, by a first controller, an electrocardiogram signal of the measurement subject based on a biopotential detected by the electrode;
measuring, by a perspiration sensor, electrical characteristics derived from perspiration of a second part of the four limbs of the measurement subject; and
calculating, by a second controller, a mental perspiration amount of the measurement subject based on the electrical characteristics measured by the perspiration sensor.

19. The method of claim 18, further comprising:
attaching a first sensor device to one of a right side portion or a left side portion of the measurement subject; and
attaching a second sensor device to the other one of the right side portion or the left side portion,
wherein the first sensor device includes a first electrode contacting the skin of one of the right side portion and the left side portion,
the second sensor device includes a second electrode contacting the skin of the other one of the right side portion and the left side portion, the first controller, the perspiration sensor, and the second controller, and
obtaining the electrocardiogram signal based on biopotentials detected by the first and second electrodes.

20. The method of claim 18, wherein
measuring the electrical characteristics comprises measuring a change in impedance of the skin of the measurement subject, and
calculating the mental perspiration amount is based on the measured change in impedance of the skin.

21. The method of claim 19, wherein
an electrode for the perspiration sensor to measure the electrical characteristics and the second electrode are common,
applying, by the perspiration sensor, a signal having a frequency different from that of the biopotential to the second electrode to measure the electrical characteristics, and
extracting, by the first controller, the biopotential by frequency filtering processing.

22. The method of claim 19, wherein
an electrode for the perspiration sensor to measure the electrical characteristics and the second electrode are common, and
performing a measurement of the electrical characteristics by the perspiration sensor and a measurement of the electrocardiogram signal by the first controller in a time-division manner.

23. The method of claim 22, further comprising:
switching, by a switch in the second sensor device, connection of the second electrode, and
controlling, by the first controller, the switch such that the second electrode and the perspiration sensor are connected at a timing when the perspiration sensor measures the electrical characteristics, and the second electrode and the first controller are connected at a timing when the first controller measures the electrocardiogram signal.

24. A biosignal measurement system comprising:
a first sensor device configured to attach to a first limb of a measurement subject;
a second sensor device configured to attach to a second limb of the measurement subject;
wherein the first sensor device includes a first electrode configured to contact skin of the first limb;
wherein the second sensor device includes:
a second electrode configured to contact skin of the second limb;
a controller configured to obtain an electrocardiogram signal of the measurement subject based on biopotentials detected by the first and second electrodes;
a perspiration sensor configured to measure electrical characteristics of the skin of the second limb; and
a processor configured to determine a perspiration level of the measurement subject based on the measured electrical characteristics.

25. The biosignal measurement system of claim 24, wherein:
the first sensor device further includes a first amplifier configured to amplify the biopotential detected by the first electrode;
the second sensor device further includes a second amplifier configured to amplify the biopotential detected by the second electrode; and
the controller is configured to obtain the electrocardiogram signal based on the amplified biopotentials from the first and second amplifiers.

26. The biosignal measurement system of claim 25, wherein:
the first sensor device further includes a first wireless transmitter configured to transmit data of the amplified biopotential from the first amplifier to the second sensor device; and
the second sensor device further includes a wireless receiver configured to receive the transmitted data from the first sensor device.

27. The biosignal measurement system of claim 24, wherein:
the perspiration sensor is configured to apply a signal to the second electrode to measure the electrical characteristics; and
the controller is configured to extract the biopotential detected by the second electrode by filtering out the applied signal.
